(19) United States
(12) Patent Application Publication
(10) Pub. No.: US 2024/0345104 A1
(43) Pub. Date: Oct. 17, 2024

(54) COMPOSITIONS AND METHODS FOR ASSAYING CIRCULATING MOLECULES

(71) Applicant: GUARDANT HEALTH, INC., Palo Alto, CA (US)

(72) Inventors: AmirAli TALASAZ, Atherton, CA (US); Andrew KENNEDY, San Diego, CA (US)

(21) Appl. No.: 18/506,573

(22) Filed: Nov. 10, 2023

Related U.S. Application Data
(63) Continuation of application No. PCT/US2022/031372, filed on May 27, 2022.
(60) Provisional application No. 63/194,789, filed on May 28, 2021.

(51) Int. Cl. G01N 33/92; C12Q 1/6886; G01N 33/53; G01N 33/574

(52) U.S. Cl. CPC G01N 33/92; C12Q 1/6886; G01N 33/5308; G01N 33/57488; C12Q 2600/154; C12Q 2600/156

(57) ABSTRACT

Provided herein are methods of detecting and quantifying target molecules associated with cell debris. Provided herein are also methods for determining the likelihood that a subject has a disease or condition, such as cancer.

### CROSS-REFERENCE TO RELATED APPLICATIONS

This application is a continuation of International PCT Application No. PCT/US2022/031372, filed May 27, 2022, which claims the benefit of priority of U.S. Provisional Patent Application No. 63/194,789, filed May 28, 2021, which are incorporated by reference herein in its entirety for all purposes.

### FIELD OF THE INVENTION

The present disclosure provides compositions and methods related to assaying circulating molecules, such as proteins from circulating cell debris. In some embodiments, the circulating proteins from cell debris are from tumor cells. In some embodiments, the proteins being assayed are from a subject having or suspected of having a disease or disorder, such as cancer.

### INTRODUCTION AND SUMMARY

Invasive diagnostic procedures, including biopsies, are commonly used for detecting or diagnosing cancer, ulcers, liver diseases, infections, transplant rejections, and other diseases and disorders in which analysis of cells or tissue from a possible site of a malady are analyzed for relevant features. Detection of diseases and disorders based on analysis of body fluids (“liquid biopsies”), such as blood, is an intriguing alternative. A liquid biopsy is noninvasive, sometimes requiring only a blood draw. However, it has been challenging to develop accurate and sensitive methods for analyzing proteins in liquid biopsy material in part because some of the same proteins released into body fluids due to disease are the same proteins that are normally present in body fluids.

Cell debris are components of dead cells that can be released into the blood or other bodily fluids following apoptosis, autophagic cell death, necrosis, or other types of cell death. For example, cell death can lead to fragmentation of cell membranes into biomolecular complexes containing cell surface proteins. Many types of diseases involve aberrant cell death or changes in cell death, including cancer, autoimmune diseases, infection and sepsis, myocardial infarction, ischemic injury, brain injury, liver disease, and neurodegenerative disease. Exosomes and other nanoscale vesicles secreted by cells, such as tumor cells, contain proteins and nucleic acids that can also provide important information about the state of the cell from which they originated. Identifying and quantifying circulating molecules, such as proteins, from cell debris and exosomes, and differentiating those molecules from soluble circulating proteins can provide important information for detection of disease in a subject.

The methods herein provide an approach to quantify and identify the levels of circulating molecules, such as proteins derived from cell debris. In some embodiments, tumor cell-derived proteins, such as cell surface proteins embedded in cell debris, are detected in blood by identifying biomolecular complexes as tumor-derived if they contain both a cell-surface protein of interest and a marker specific for dead or dying cells or fragments thereof, e.g., cell membrane debris. In some embodiments, methods herein may be performed in a single step, for example, by detecting a signal generated by the presence of both a cell surface protein and a component of cell debris; or sequentially, for example, by enriching a sample for a marker of cell debris, then assaying the enriched sample for a secondary marker, e.g., a protein of interest. In certain embodiments, methods herein facilitate the measurement of tumor tissue diagnostic markers in the blood, e.g., with improved specificity and/or sensitivity by focusing on markers associated with cell debris, and, in some embodiments, provide an alternative to immunohistochemistry assays of biopsied tissues. Applications of the methods herein also include profiling cell type distribution of dead or dying cells in blood and identifying circulating protein signatures that can be reflective of certain disease states.

The methods herein may include additional steps that can provide information about DNA variations and modifications, including but not limited to epigenetic and sequence variations in cfDNA or nucleic acids isolated from exosomes. Such methods comprising protein and DNA analysis may provide even more improved information about the likelihood of a particular disease state of a subject.

The present disclosure aims to meet the need for improved analysis of molecules originating from dead or dying cells, such as from tumor cells. Improved detection of cancer markers in blood allows for more accurate detection of disorders (diagnosis) and therefore improved treatments. Accordingly, the following exemplary embodiments are provided.

Embodiment 1 is a method of detecting a cell debris-associated target molecule in a sample, the method comprising:
a) contacting the sample or a subsample thereof with at least one binding molecule, wherein the at least one binding molecule binds a cell debris marker, thereby producing complexes comprising the at least one binding molecule and cell debris, the cell debris comprising a membrane fragment; and
b) detecting the presence or level of at least one target molecule associated with the complexes.

Embodiment 1.1 is the method of embodiment 1, wherein the sample or subsample thereof is contacted with a plurality of different binding molecules, wherein the binding molecules bind a plurality of cell debris markers or at least one cell debris marker and at least one target molecule.

Embodiment 1.2 is the method of any one of the preceding embodiments, wherein the cell debris comprises a plasma membrane fragment.

Embodiment 1.3 is the method of any one of the preceding embodiments, wherein the cell debris comprises an inner membrane fragment.

Embodiment 1.4 is the method of any one of the preceding embodiments, wherein the cell debris comprises an inner plasma membrane fragment.

Embodiment 2 is the method of any one of the preceding embodiments, wherein the sample is obtained from a subject.

Embodiment 3 is the method of any one of the preceding embodiments, wherein the sample is a blood sample.

Embodiment 4 is the method of embodiment 3, wherein the blood sample is a whole blood sample.

Embodiment 5 is the method of embodiment 3, wherein the blood sample is a plasma sample.

Embodiment 6 is the method of embodiment 3, wherein the blood sample is a plasma pellet sample or a buffy coat sample.

Embodiment 7 is the method of any one of the preceding embodiments, wherein at least one binding molecule is a protein, wherein the protein is optionally an antibody.

Embodiment 8 is the method of any one of the preceding embodiments, wherein at least one binding molecule binds a cell debris marker.

Embodiment 9 is the method of the immediately preceding embodiment, wherein the cell debris marker is an inner membrane marker.

Embodiment 10 is the method of any one of embodiments 8-9, wherein the cell debris marker is phosphatidylserine or phosphatidylethanolamine.

Embodiment 11 is the method of any one of embodiments 8-10, wherein the cell debris marker is phosphatidylserine.

Embodiment 12 is the method of the immediately preceding embodiment, wherein at least one binding molecule is Annexin V or an antibody specific for phosphatidylserine.

Embodiment 13 is the method of embodiment 10, wherein the cell debris marker is phosphatidylethanolamine.

Embodiment 14 is the method of the immediately preceding embodiment, wherein at least one binding molecule is an antibody specific for phosphatidylethanolamine.

Embodiment 15 is the method of any one of the preceding embodiments, wherein the at least one binding molecule comprises a label or is conjugated to a solid support.

Embodiment 16 is the method of the immediately preceding embodiment, wherein the at least one binding molecule comprises a label and the method further comprises capturing the at least one binding molecule by binding the label to a solid support.

Embodiment 17 is the method of embodiment 15, wherein the at least one binding molecule is conjugated to a label, wherein the label comprises a fluorophore, biotin, a peptide, or an oligonucleotide.

Embodiment 17.1 is the method of embodiment 15, wherein the at least one binding molecule is conjugated to an oligonucleotide.

Embodiment 18 is the method of any one of embodiments 15-16, wherein the solid support comprises a bead.

Embodiment 19 is the method of the immediately preceding embodiment, where the at least one binding molecule is conjugated to a magnetic bead.

Embodiment 20 is the method of any one of the preceding embodiments, wherein the method comprises capturing the complexes from the sample or subsample thereof prior to the detecting.

Embodiment 21 is the method of the immediately preceding embodiment, wherein the capturing comprises separating components of the sample or subsample thereof that are not bound to the at least one binding molecule from the complexes to which the at least one binding molecule is bound.

Embodiment 22 is the method of any one of embodiments 20-21, wherein the detecting comprises mass spectrometric analysis of target molecules associated with the complexes.

Embodiment 23 is the method of any one of embodiments 20-21, wherein the detecting comprises contacting the complexes with at least one binding molecule that binds a target molecule potentially associated with the complexes.

Embodiment 24 is the method of the immediately preceding embodiment, wherein at least one binding molecule that binds a target molecule associated with the complexes is an antibody specific for a target molecule.

Embodiment 25 is the method of any one of embodiments 23-24, wherein at least one binding molecule that binds a target molecule comprises a label.

Embodiment 26 is the method of the immediately preceding embodiment, wherein the label is a fluorophore or an oligonucleotide.

Embodiment 27 is the method of the immediately preceding embodiment, wherein the label is an oligonucleotide.

Embodiment 28 is the method of the immediately preceding embodiment, wherein the label is an oligonucleotide, and the binding molecule that binds a cell debris marker comprises an oligonucleotide.

Embodiment 28.1 is the method of any one of embodiments 17, 17.1, 27, or 28, wherein the oligonucleotide or oligonucleotides comprise DNA.

Embodiment 28.2 is the method of any one of embodiments 17, 17.1, 27, or 28, wherein the oligonucleotide or oligonucleotides comprise RNA.

Embodiment 28.3 is the method of any one of embodiments 17, 17.1, 27, 28, 28.1, or 28.2, wherein the oligonucleotide or oligonucleotides are at least partially single stranded.

Embodiment 28.4 is the method of any one of embodiments 17, 17.1, 27, 28, 28.1, 28.2, or 28.3, wherein the oligonucleotide or oligonucleotides have a length of at least 10, 11, 12, 13, 14, 15, 16, 17, or 18 nucleotides.

Embodiment 28.5 is the method of any one of embodiments 17, 17.1, 27, 28, 28.1, 28.2, 28.3, or 28.4, wherein the oligonucleotide or oligonucleotides independently have a length of 10, 11, 12, 13, 14, 15, 16, 17, 18, 19, 20, 21-25, 26-30, 31-40, or 41-50 nucleotides.

Embodiment 29 is the method of any one of embodiments 28-28.5, wherein the detecting comprises a proximity ligation assay or a proximity extension assay.

Embodiment 29.1 is the method of any one of embodiments 28-29, wherein the oligonucleotides comprise complementary hybridization sequences which are 3′ of a tag (e.g., a molecular barcode, e,g., which identifies the binding molecule with which the label was associated and optionally provides additional information, e.g., for identifying the sample and/or pre-enriched fraction being analyzed).

Embodiment 29.2 is the method of any one of embodiments 28-29.1, wherein when oligonucleotides comprising complementary hybridization sequences are in proximity (e.g., when the binding molecules to which the oligonucleotides are attached are bound to the same piece of cell debris), the hybridization sequences hybridize to each other, forming a substrate for extension by a DNA polymerase.

Embodiment 29.3 is the method of embodiment 29.2, wherein the substrate for extension by a DNA polymerase is extended to produce an extended product and the extended product is detected (e.g., by sequencing or qPCR, either of which may follow amplification and/or library preparation steps), thus indicating the presence of the target molecule associated with cell debris.

Embodiment 29.4 is the method of any one of embodiments 28-29, wherein the oligonucleotides comprise tags (e.g., a molecular barcode, e,g., which identifies the binding molecule with which the label was associated and optionally provides additional information, e.g., for identifying the sample and/or pre-enriched fraction being analyzed).

Embodiment 29.5 is the method of any one of embodiments 28-29 or 29.4, wherein a ligation template and ligase are provided that result in ligation of oligonucleotides to each other, forming a ligation product, if the oligonucleotides are in proximity (e.g., when the binding molecules are bound to the same piece of cell debris).

Embodiment 29.6 is the method of embodiment 29.5, further comprising amplifying the ligation product.

Embodiment 29.7 is the method of embodiment 29.5 or 29.6, further comprising detecting the ligation product or an amplification product thereof (e.g., by sequencing or qPCR, which may follow amplification and/or library preparation steps), thus indicating the presence of the target molecule associated with cell debris.

Embodiment 30 is the method of any one of embodiments 20-29.7, wherein the detecting comprises an immunoassay.

Embodiment 31 is the method of the immediately preceding embodiment, wherein the immunoassay is an enzyme-linked immunosorbent assay, a sandwich assay, an electrochemiluminescent assay, or a multiplex immunoassay.

Embodiment 32 is the method of embodiments 20-31, wherein the detecting comprises flow cytometric analysis of the complexes.

Embodiment 33 is the method of any one of the preceding embodiments, wherein a plurality of target molecules associated with the complexes are detected.

Embodiment 34 is the method of the immediately preceding embodiment, wherein the plurality of target molecules is 2 to 10,000, 2 to 5,000, 2 to 1,000, or 2 to 100 target molecules.

Embodiment 35 is the method of any one of embodiments 1-19 or 33-34, wherein the method comprises capturing the complexes after contacting the complexes with at least one binding molecule that binds a target molecule potentially associated with the complexes.

Embodiment 35.1 is the method of any one of embodiments 1-19 or 33-34, wherein the sample is contacted with at least one binding molecule that binds a target molecule before contacting the sample with the cell debris marker binding molecule.

Embodiment 35.2 is the method of any one of embodiments 1-19 or 33-34, wherein the sample is contacted with at least one binding molecule that binds a target molecule after contacting the sample with the cell debris marker binding molecule.

Embodiment 35.3 is the method of any one of embodiments 1-19 or 33-34, wherein the sample is contacted with at least one binding molecule that binds a target molecule at the same time as contacting the sample with the cell debris marker binding molecule.

Embodiment 36 is the method of any one of the preceding embodiments, wherein at least one target molecule, two or more of the plurality of target molecules, or each of the plurality of target molecules is a protein.

Embodiment 37 is the method of any one of the preceding embodiments, wherein at least one target molecule, two or more of the plurality of target molecules, or each of the plurality of target molecules is a carbohydrate, optionally a glycoprotein carbohydrate.

Embodiment 38 is the method of any one of the preceding embodiments, wherein at least one target molecule is a molecule associated with a disease, two or more of the plurality of target molecules is a molecule associated with a disease, or each of the plurality of target molecules is a molecule associated with a disease.

Embodiment 39 is the method of the immediately preceding embodiment, wherein the disease is cancer.

Embodiment 40 is the method of the immediately preceding embodiment, wherein the at least one target molecule is upregulated in tumor cells relative to healthy cells of the same tissue type.

Embodiment 41 is the method of any one of embodiments 38-40, wherein at least one, two or more, or each of the target molecules is selected from PD-L1, CTLA4, NYESO1, mesothelin, CA15-3, CA19-9, CA-125, and CA-172-4.

Embodiment 42 is the method of any one of the preceding embodiments, wherein at least one target molecule, two or more target molecules, or each of the plurality of target molecules is a cell type marker.

Embodiment 43 is the method of the immediately preceding embodiment, wherein the cell type markers are selected from markers for immune cells and solid tissue cells.

Embodiment 44 is the method of the immediately preceding embodiment, wherein the cell type markers are selected from markers for colon, lung, breast, skin, prostate, stomach, pancreas, and liver cell type markers.

Embodiment 45 is the method of any one of the preceding embodiments, wherein the sample is obtained from a subject having a disease, and the detecting comprises identifying a plurality of target molecules, wherein the identifying comprises mass spectrometric analysis of target proteins.

Embodiment 46 is the method of any one of the preceding embodiments, wherein the method comprises measuring total cell debris levels in the sample or subsample thereof.

Embodiment 46.1 is the method of the immediately preceding embodiment, wherein the total cell debris levels are measured by quantifying at least one cell debris marker in the sample or a subsample thereof.

Embodiment 47 is the method of embodiment 46 or 46.1, wherein the sample is obtained from a subject having a disease, and wherein the total cell debris levels is measured relative to total cell debris levels in a sample or subsample thereof obtained from a healthy individual.

Embodiment 48 is the method of any one of any one of the preceding embodiments, wherein the method comprises analyzing DNA in a subsample of the sample or in a second sample obtained from the same subject from which the first sample is obtained.

Embodiment 49 is the method of the immediately preceding embodiment, wherein the subsample or second sample is a plasma or serum sample.

Embodiment 50 is the method of the immediately preceding embodiment, wherein the DNA is cfDNA.

Embodiment 50.1 is the method of any one of embodiments 48-50, wherein analyzing DNA comprises quantifying at least one epigenetic feature of target regions of DNA, optionally wherein the epigenetic feature comprises methylation.

Embodiment 50.2 is the method of any one of embodiments 48-50, wherein analyzing DNA comprises detecting or quantifying one or more genetic variants in one or more target regions of DNA.

Embodiment 51 is a method of detecting the presence or absence of cancer, comprising performing the method of any one of the preceding embodiments, wherein the presence or level of at least one target molecule associated with the complexes is indicative of the presence or absence of cancer.

Embodiment 52 is a method of screening for cancer, comprising performing the method of any one of embodiments 1-50.2 on samples from a plurality of subjects, wherein the presence or level of at least one target molecule associated with the complexes indicates that the corresponding subject may have cancer.

Embodiment 53 is a method of monitoring residual cancer or detecting the presence or absence of recurrent cancer, comprising performing the method of any one of embodiments 1-50.2, wherein the presence or level of at least one target molecule associated with the complexes is indicative of the status of the cancer or the presence or absence of recurrent cancer.

Embodiment 54 is a method of identifying a therapy for treating a disease, optionally wherein the disease is a cancer, the method comprising performing the method of any one of embodiments 1-50.2, wherein the presence or level of at least one target molecule associated with the complexes is indicative of a suitable therapy for treating a disease.

### BRIEF DESCRIPTION OF THE DRAWINGS

FIG. 1A-B show exemplary workflows of methods disclosed herein. At least a portion of a whole blood sample is fractionated into plasma, buffy coat, and red blood cells. The plasma is fractionated into a plasma pellet and plasma supernatant. In FIG. 1A, cell debris is isolated from the buffy coat fraction and plasma pellet and/or from a portion of the whole blood sample. Exosomes can also be isolated from the plasma supernatant and/or from a portion of the whole blood sample. Target proteins associated with the cell debris and/or exosomes are detected. In another exemplary method, shown in FIG. 1B, the buffy coat fraction, plasma pellet, plasma supernatant, and/or a portion of the whole blood sample are contacted with antibodies specific for a marker or a target protein that are linked to oligonucleotides that facilitate hybridization and extension if the marker and target protein are in proximity, followed by strand extension, amplification, and sequencing to detect the target proteins. In another embodiment, the oligonucleotides can be configured to facilitate ligation if the marker and target protein are in proximity, followed by strand extension, amplification, and sequencing to detect the target proteins.

FIG. 2 is a schematic diagram of an example of a system suitable for use with some embodiments of the disclosure.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

“Cell debris marker” as used herein means a molecule, such as a protein, lipid, or carbohydrate, that is physically associated with or embedded in a component of a dead or dying cell and is present in greater proportion in such components of ruptured or intact dead or dying cells than on the outer membrane of intact live cells, intact vesicles, or in the soluble fraction of a sample. The component of the dead or dying cell associated with the cell debris marker may be dissociated from other components of the cell from which it originated or may be contained in an intact dead or dying cell. Examples of cell debris markers include but are not limited to molecules associated with or localized to the inner plasma membrane, e.g., phosphatidylserine and phosphatidylethanolamine.

A “dying cell” as used herein is an intact pre-apoptotic, pre-necrotic, or autophagic cell in which physical changes associated with cell death have begun to occur, e.g., shuttling of internal phospholipids to the external side of the plasma membrane.

“Cell debris” as used herein means components of dead cells that can be released into the blood or other bodily fluids following apoptosis, autophagic cell death, necrosis, or other types of cell death. For example, cell death can lead to fragmentation of cell membranes into biomolecular complexes containing cell surface proteins. In some embodiments, cell debris comprises membrane fragments released from a dead or dying cell and associated molecules such as proteins and/or carbohydrates.

“Cell debris marker binding molecule” and a “binding molecule” that “binds a cell debris marker” as used herein means a molecule that specifically binds a cell debris marker. For example, an antibody that specifically binds a cell debris marker is a cell debris marker binding molecule. Examples of cell debris marker binding molecules include but are not limited to Annexin V, an antibody for phosphatidylserine, and an antibody for phosphatidylethanolamine. Binding molecules also include nanobodies, aptamers, affimers, DARPins, and the like.

A first molecule is “associated with” a complex or other molecule if the first molecule is bound to the complex or other molecule directly or indirectly (e.g., through a chain of one or more additional molecules).

“Exosome marker” as used herein means a molecule, such as a protein, lipid, or carbohydrate, that is physically associated with or embedded in the outer membrane of an exosome and is present in greater proportion in exosomes than on the outer membrane of intact live cells, cell debris, or in the soluble fraction of a sample. Examples of exosome markers include but are not limited to tetraspanines, CD9, CD63, and CD81.

“Exosome marker binding molecule” as used herein means a molecule that specifically binds an exosome marker. For example, an antibody that specifically binds an exosome marker is an exosome marker binding molecule. Binding molecules also include nanobodies, aptamers, affimers, DARPins, and the like.

“Cell type marker” as used herein means a molecule that is present in higher proportion in one or more cell types than in other cell types present in the same sample or than in any other cell type.

“Solid tissue cells” as used herein means cells in or derived from a solid tissue. Solid tissue cells exclude circulating cell types, such as cells normally present in blood or lymph. Examples of solid tissue cell types include but are not limited to colon, lung, breast, skin, prostate, stomach, pancreas, and liver cells.

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, a “blood sample” refers to a sample comprising whole blood or a component thereof (e.g., plasma, serum, buffy coat, plasma pellet).

As used herein, “partitioning” of nucleic acids, such as DNA molecules, means separating, fractionating, sorting, or enriching a sample or population of nucleic acids into a plurality of subsamples or subpopulations of nucleic acids based on one or more modifications or features that is in different proportions in each of the plurality of subsamples or subpopulations. Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, the form of the “originally isolated” sample refers to the composition or chemical structure of a sample at the time it was isolated and before undergoing any procedure that changes the chemical structure of the isolated sample. Similarly, a feature that is “originally present” in a molecule refers to a feature present in an “original molecule” or in molecules “originally comprising” the feature before the molecule undergoes any procedure that changes the chemical structure of the molecule.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

“Capturing” one or more target molecules, such as one or more proteins or nucleic acids or one or more molecules comprising at least one target region refers to preferentially isolating or separating the one or more target molecules from non-target molecules.

As used herein, a “label” is a capture moiety, fluorophore, oligonucleotide, or other moiety that facilitates detection, separation, or isolation of that to which it is attached.

As used herein, a “capture moiety” is a molecule that allows affinity separation of molecules linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “tag” is a molecule, such as a nucleic acid, label, fluorophore, or peptide, containing information that indicates a feature of the molecule to which the tag is associated. For example, molecules can bear a sample tag (which distinguishes molecules in one sample from those in a different sample), a molecular tag/molecular barcode/barcode (which distinguishes different molecules from one another (in both unique and non-unique tagging scenarios), a partition tag (which distinguishes molecules in a partition from molecules in another partition) a purification tag, and/or a detectable tag or label.

As used herein, a “target molecule” is a molecule, such as a protein, carbohydrate, or lipid, the presence or absence of which is detected. The identity of target molecule need not be known before the detection. The identity of a target molecule may be determined as part of a method described herein, for example, by using mass spectrometry to analyze a target protein.

“Specifically binds” in the context of a primer, probe, or other oligonucleotide, a protein, or other binding molecule and a target sequence means that under appropriate hybridization conditions, the primer, oligonucleotide, or probe hybridizes to its target sequence, or replicates thereof, to form a stable hybrid, while at the same time formation of stable non-target hybrids is minimized. Thus, a primer or probe hybridizes to a target sequence or replicate thereof to a sufficiently greater extent than to a non-target sequence, to ultimately enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, N Y, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Plasma pellet” as used herein means the precipitated material following centrifugation of plasma that was previously separated from whole blood. The plasma may be separated from whole blood by a first centrifugation, and the plasma pellet may be generated by a second centrifugation of only the plasma portion (supernatant) of the first centrifugation. The supernatant of the second centrifugation may be referred to as isolated plasma, and the precipitate is a plasma pellet. In some embodiments, a plasma pellet comprises cell debris (e.g., of a generally lower mass or smaller size than cell debris found in the buffy coat). In some embodiments, the plasma pellet is substantially free of cfDNA, cfRNA, soluble proteins, exosomes and metabolites. “Substantially free” means free to a sufficient extent that the relevant properties are not meaningfully impacted by the presence of a minor impurity.

“Immunoassay” as used herein means an assay or method comprising contacting a molecule or sample with an antibody in order to test the function or detect, identify, and/or quantify the presence of one or more components of the sample. Examples of immunoassays may include but are not limited to enzyme-linked immunosorbent assays (ELISAs), sandwich assays, eletrochemiluminescence (ECL) assays, and multiplex assays.

An “antibody” as used herein is used broadly encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

A protein or nucleic acid is “produced by a tumor” if it originated from a tumor cell. DNA that originated from a tumor cell is “circulating tumor DNA” (“ctDNA”). Tumor cells are neoplastic cells that originated from a tumor, regardless of whether they remain in the tumor or become separated from the tumor (as in the cases, e.g., of metastatic cancer cells and circulating tumor cells).

A “target region” in the context of a nucleic acid refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity).

“Sequence-variable target regions” refer to target regions that may exhibit changes in sequence such as nucleotide substitutions (i.e., single nucleotide variations), insertions, deletions, or gene fusions or transpositions in neoplastic cells (e.g., tumor cells and cancer cells) relative to normal cells. A sequence-variable target region set is a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, or 2 nucleotides, or that affect 1 nucleotide.

“Epigenetic target regions” refers to target regions that may show sequence-independent differences in different cell or tissue types (e.g., different types of immune cells) or in neoplastic cells (e.g., tumor cells and cancer cells) relative to normal cells; or that may show sequence-independent differences in DNA, such as cfDNA, from different cell types or from subjects having cancer relative to DNA, such as cfDNA, from healthy subjects, or in cfDNA originating from different cell or tissue types that ordinarily do not substantially contribute to cfDNA (e.g., immune, lung, colon, etc.) relative to background cfDNA (e.g., cfDNA that originated from hematopoietic cells). Examples of sequence-independent changes include, but are not limited to, changes in methylation (increases or decreases), nucleosome distribution, cfDNA fragmentation patterns, CCCTC-binding factor (“CTCF”) binding, transcription start sites, and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An epigenetic target region set is a set of epigenetic target regions.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

The term “methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′->3′ direction of the nucleic acid sequence). In some embodiments, DNA methylation refers to addition of a methyl group to adenine, such as in N⁶-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5 mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### II. Exemplary Methods

### A. Cell-Debris Associated Target Molecule Identification and Quantification

Methods disclosed herein comprise steps of contacting a sample or subsample with at least one cell debris marker binding molecule and detecting the presence or level of at least one target molecule, such as a target protein, associated with the cell debris. The cell debris comprises a membrane fragment. The target molecule is different from the cell debris marker. Detecting a target molecule associated with cell debris can be more informative than detecting the target molecule in a sample of, e.g., whole blood or plasma, e.g., because the latter may show a higher background or baseline level of the target protein, whereas when a subject is healthy, the level of the target molecule associated with cell debris will be low. In some embodiments, at least one target molecule, such as a target protein, associated with the cell debris is quantified. In some embodiments, a post-translational modification of a target protein associated with the cell debris is detected or quantified.

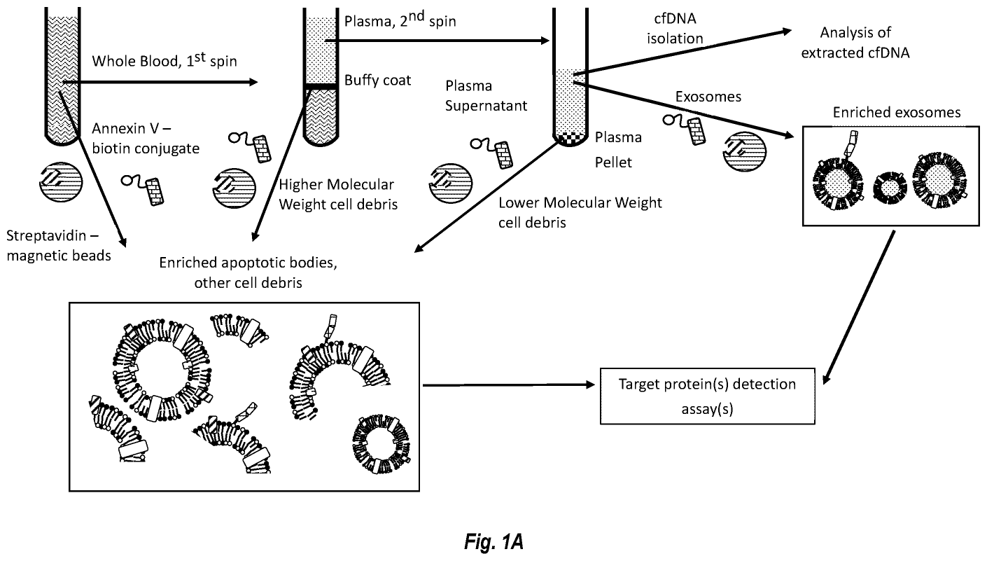

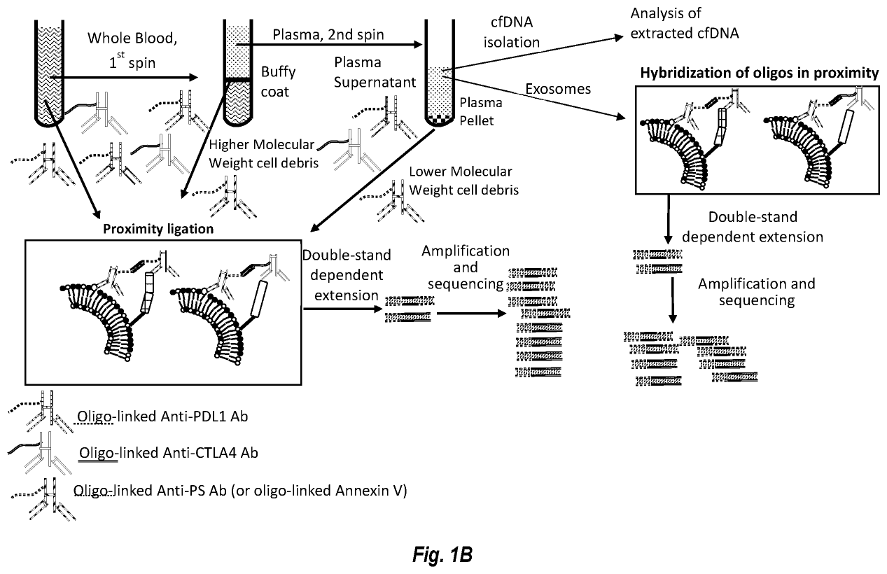

As illustrated in FIG. 1A, cell debris can be enriched or isolated from a whole blood sample or from various subsamples thereof, e.g., prepared by centrifugation. In some embodiments, one or more, or each, of a whole blood sample, a buffy coat fraction, and a plasma pellet are contacted with at least one cell debris marker binding molecule. An exemplary cell debris marker binding molecule is annexin V or a conjugate thereof, e.g., to biotin. Other exemplary cell debris marker binding molecules are described elsewhere herein. One or more target molecules, such as proteins, can be detected or quantified in the enriched cell debris, which may comprise apoptotic bodies. In some embodiments, exosomes are also enriched from the sample or a subsample thereof, e.g., from supernatant following a second centrifugation of plasma. One or more target molecules, which where applicable may be the same or different as the one or more target molecules detected or quantified in the enriched cell debris, can be detected or quantified in the enriched exosomes. In some embodiments, cell-free DNA is isolated from a sample or subsample thereof, e.g., from supernatant following a second centrifugation of plasma. The cell-free DNA can be analyzed to detect or quantify sequence variations or epigenetic features, e.g., as described elsewhere herein.

The contacting and detecting can be performed sequentially or simultaneously. In some embodiments, sequential methods comprise enriching, capturing, or isolating complexes comprising the at least one cell debris marker binding molecule, the at least one cell debris marker, and one or more target molecules and subsequently detecting the one or more target molecules. In some embodiments, methods comprise simultaneously contacting the sample or subsample with at least one cell debris marker binding molecule and at least one binding molecule specific for a target molecule, wherein the binding molecules are configured to facilitate direct detection of target molecules that are associated with cell debris, such as a proximity ligation assay or proximity extension assay. See, for example, the proximity ligation assay shown in FIG. 1B. For example, FIG. 1B illustrates detecting or quantifying PDL1 and/or CTLA4 in cell debris using oligo-linked antibodies or conjugates (e.g., antibodies specific for one or both of PDL1 and CTLA4 and Annexin V or an antibody specific for phosphatidylserine). Optionally, exosomes can also be analyzed using a similar proximity ligation or proximity extension approach with plasma supernatant, and/or cell-free DNA can be analyzed as discussed above with respect to FIG. 1A.

In a proximity extension assay, first and second binding molecules (e.g., a cell debris marker binding molecule and a target binding molecule) are labeled with oligonucleotides that comprise complementary hybridization sequences which are 3′ of a tag (e.g., a molecular barcode, which identifies the binding molecule with which the label was associated; the tag may further comprise one or more additional elements to provide additional information, e.g., regarding the sample, such as a sample tag, and/or pre-enriched fraction being analyzed, such as a partition tag; this can facilitate subsequent pooling). The tags may have any of the features described elsewhere herein with respect to tags. When the oligonucleotides are in proximity (as occurs, e.g., when the binding molecules are bound to the same piece of cell debris), the hybridization sequences can hybridize to each other, forming a substrate for extension by a DNA polymerase at an above-background rate. The extended product can then be detected (e.g., by sequencing or qPCR, which may follow amplification and/or library preparation steps), thus indicating the presence of the target molecule associated with cell debris. For a general example of a proximity extension assay, see WO2007/005649 A2 to Anderson et al., which is incorporated herein by reference for all purposes. Additionally, amplification of a nucleic acid label attached to an antibody is also discussed in U.S. Pat. No. 5,665,539 to Sano et al., which is incorporated herein by reference for all purposes.

In a proximity ligation assay, first and second binding molecules (e.g., a cell debris marker binding molecule and a target binding molecule) are labeled with oligonucleotides. A ligation template and ligase are provided that result in ligation of oligonucleotides to each other at an above-background rate if they are in proximity (as occurs, e.g., when the binding molecules are bound to the same piece of cell debris). The ligation product can comprise at least a portion of the ligation template and/or be at least partially double-stranded, e.g., through hybridization of a portion of the ligation template and/or a complementary strand synthesis step using an appropriate primer. The oligonucleotides may include tags and/or barcodes as discussed above and as described elsewhere herein. The tags may have any of the features described elsewhere herein with respect to tags. The ligation product can be a substrate for amplification. The ligation product can be detected (e.g., by sequencing or qPCR, which may follow amplification and/or library preparation steps), thus indicating the presence of the target molecule associated with cell debris. General examples of proximity ligation assays may be found in U.S. Pat. No. 9,518,296 B2 to Ruff et al. and US2007/0281367 A1 to Hennessy et al., both of which are incorporated herein by reference for all purposes.

Also disclosed are methods in which an exosome marker binding molecule is used in place of or in combination with the cell debris marker binding molecule, such that the methods can comprise enriching, capturing, or isolating complexes comprising the at least one exosome marker binding molecule, the at least one exosome marker, and one or more target molecules and subsequently detecting the one or more target molecules from the exosome, e.g., in addition to the one or more target molecules in complexes comprising a cell debris marker. Exosomes are not considered cell debris.

In some embodiments, the methods comprise contacting the sample or subsample with a cell debris marker binding molecule and detecting one or more target molecules, such as target proteins, associated with the cell debris. In some embodiments, the cell debris marker is an inner membrane marker. Without wishing to be bound by theory, lipids that are nearly exclusively localized to the inner plasma membrane leaflet are flipped to the outer membrane leaflet in pre-apoptotic, apoptotic, and other dying and dead cells. Additionally, ruptured cell components comprise inner membrane leaflets that are exposed to sample solvent. Therefore, selective binding inner membrane markers may facilitate selective binding to cell debris and therefore selective detection of target molecules associated with the cell debris over soluble proteins.

Exemplary inner membrane markers include but are not limited to phosphatidylserine and phosphatidylethanolamine. In some embodiments, the cell debris marker binding molecule is a protein, such as an antibody, nanobody, affimer, or DARpin that specifically binds a cell debris marker. In some embodiments, the protein is Annexin V or an antibody specific for phosphatidylserine. In some embodiments, the cell debris marker binding molecule is a nucleic acid, such as an aptamer. In some embodiments, the cell debris marker binding molecule comprises a label, such as a capture moiety (e.g., biotin) or an oligonucleotide.

In some embodiments, the methods comprise measuring a total cell debris level in the sample or a subsample thereof. Total cell debris levels may be measured, e.g., by quantifying the total amount of a cell debris marker, which may be any of those described herein. The cell debris marker used for measuring the total cell debris level may be the same as or different from the cell debris marker bound by the binding molecule. Where more than one cell debris marker binding molecule is used, the cell debris marker used for measuring the total cell debris level may be the same as one of the cell debris markers bound by a binding molecule, or may be different from all of the cell debris markers bound by a binding molecule. Any suitable measurement technique may be used for such measurement (e.g., immunoassay, mass spectrometry, etc.).

The methods herein may also be used to assay exosomes. In such embodiments, the methods comprise contacting the sample or subsample with at least one exosome marker binding molecule and detecting the presence or level of at least one target molecule, such as a target protein, associated with the exosome. In some embodiments, the exosome marker is a transmembrane protein that is present in greater proportion in exosome membranes than in other membranes in the sample. In some embodiments, the exosome marker is a tetraspanine. In some embodiments, the exosome marker is CD9, CD63, or CD81. In some embodiments, the exosome marker binding molecule is a protein, such as an antibody, nanobody, affimer, or DARpin that specifically binds an exosome marker. In some embodiments, the protein is an antibody specific for CD9, CD63, or CD81. In some embodiments, the exosome marker binding molecule is a nucleic acid, such as an aptamer. In some embodiments, the exosome marker binding molecule comprises a label or capture moiety, such as biotin.

The methods herein comprise detecting at least one target molecule, such as a target protein. In some embodiments, the identity of the one or more target molecules is not known prior to beginning the method. In some such embodiments, the methods comprise detecting target proteins using mass spectrometric analysis and identification of the target proteins. In some embodiments, the detecting comprises contacting the sample with binding molecules specific for target molecules suspected to be present in the sample. In some embodiments, the identity of the one or more target molecules is known prior to beginning the method, and the target molecule detection methods are chosen accordingly. In some embodiments, the detecting comprises performing an immunoassay, such as an ELISA, a sandwich assay, an electrochemiluminescent (ECL) assay, or a multiplex immunoassay. In some embodiments, the detecting comprises flow cytometry analysis of the sample.

In some embodiments, the one or more target molecules are molecules derived from tumor cells, cells in another disease state, or cells that altered due to the presence of a disease in the subject from which the cells are obtained. In some embodiments, one or more target molecules are derived from cell types not normally present in the type of bodily sample obtained from a subject. In some embodiments, at least one target molecules is a target protein. In some embodiments, at least one target molecule is a target carbohydrate, such as a glycoprotein carbohydrate. In some embodiments, one or more target molecules are selected from PD-L1, CTLA4, NYESO1, mesothelin, CA15-3, CA19-9, CA-125, and CA-172-4. In some embodiments, one or more target molecules is a cell type marker, such as an immune cell type marker or solid tissue cell type marker. In some embodiments, the solid tissue cell type marker is a marker present in colon, lung, breast, skin, prostate, stomach, pancreas, or liver cells.

In some embodiments, determining the levels of target molecules facilitates disease diagnosis or identification of appropriate treatments. In some embodiments, the presence of or a change in the levels of one or more target molecules is indicative of the presence of a disease or disorder in a subject, such as cancer, precancer, an infection, transplant rejection, or other disorder that causes changes in cell death. In some embodiments, methods described herein further comprise detecting genetic variants, e.g., in a sequence-variable target region set. In some embodiments, methods described herein further comprise detecting epigenetic features, e.g., DNA methylation and/or fragmentation. In some embodiments, methods described herein further comprise detecting genetic variants, e.g., in a sequence-variable target region set, and detecting epigenetic features, e.g., genome methylation and/or fragmentation. Detection of epigenetic features may be performed in an epigenetic target region set. Exemplary sequence-variable target region sets and epigenetic target region sets are described, e.g., in WO2020/160414, published Aug. 6, 2020, which is incorporated herein by reference. Detection of genetic variants and/or epigenetic features may be performed using nucleic acids (e.g., cfDNA) from the same sample as is used for determining the levels of target molecules. Exemplary approaches for detecting genetic variants and/or epigenetic features are described elsewhere herein, including in section II.D below. In some embodiments, detection of target molecules in combination with cfDNA analysis of sequence-independent changes in epigenetic target regions, for example, cfDNA analysis as described herein, are indicative of the presence of a disease or disorder in a subject, such as cancer, precancer, an infection, transplant rejection, or other disorder that causes changes in the relative amounts of target molecules associated with cell debris and/or exosomes and DNA changes relative to a healthy subject.

### B. Subjects

In some embodiments, the sample is obtained from a subject having a cancer or a precancer, an infection, transplant rejection, or other disease directly or indirectly affecting the immune system. In some embodiments, the sample is obtained from a subject suspected of having a cancer or a precancer, an infection, transplant rejection, or other disease directly or indirectly affecting the immune system. In some embodiments, the sample is obtained from a subject having a tumor. In some embodiments, the sample is obtained from a subject suspected of having a tumor. In some embodiments, the sample is obtained from a subject having neoplasia. In some embodiments, the sample is obtained from a subject suspected of having neoplasia. In some embodiments, the sample is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

### C. Analysis

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation, rare mutations, or target molecules detected in a subject's blood if the treatment is successful as more cancers may die and shed DNA and cell debris. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In some embodiments, a method described herein comprises identifying the presence of target molecules and/or DNA produced by a tumor (or neoplastic cells, or cancer cells) or by precancer cells. In some embodiments, a method described herein comprises determining the level of target molecules and/or identifying the presence of DNA produced by a tumor (or neoplastic cells, or cancer cells) or by precancer cells. In some embodiments, determining the level of target molecules comprises determining either an increased level or decreased level of target molecules, wherein the increased or decreased level of target molecules is determined by comparing the level of target molecules with a threshold level/value.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a profile of extracellular molecules derived from the subject, wherein the profile comprises a plurality of data resulting from target molecule detections and copy number variation and rare mutation analyses. In some embodiments, an abnormal condition is cancer or precancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate a profile, fingerprint or set of data that is a summation of information derived from different cells in a heterogeneous disease. This set of data may comprise target molecule identities and levels, copy number variation, epigenetic variation, or other mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

D. Analysis of DNA; Partitioning the Sample into a Plurality of Subsamples

In some embodiments described herein, the disclosed methods further comprise analyzing DNA in a sample (which may be a separate sample from the same subject or the same sample). For example, analyzing DNA such as cell-free DNA in combination with analyzing target molecules associated with cell debris may improve the specificity and/or sensitivity of methods that detect abnormal states, such as the presence of a disease. As illustrated in FIGS. 1A-B, DNA such as cell-free DNA can be isolated from a blood sample or subsample thereof, such as a plasma supernatant obtained following centrifugation of plasma. Analyzing DNA may comprise detecting or quantifying DNA of interest. Analyzing DNA can comprise detecting genetic variants and/or epigenetic features (e.g., DNA methylation and/or DNA fragmentation).

In any of these embodiments, methylation levels can be determined using partitioning, methylation-sensitive conversion such as bisulfite conversion, direct detection during sequencing, methylation-sensitive restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated.

Detecting aberrant features in DNA (whether sequence-based, epigenetic, or both) while also detecting aberrant levels of one or more target molecules in cell debris and/or exosomes may provide greater specificity and/or sensitivity for identifying an abnormal state than detecting the DNA features alone or levels of one or more target molecules in cell debris and/or exosomes alone.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids.

Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner® Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

In some embodiments, methylation is detected using a methylation-sensitive conversion. Examples of such conversion techniques include bisulfite conversion, which converts unmodified cytosine and certain modified cytosines (e.g. 5-formyl cytosine (fC) or 5-carboxylcytosine (caC)) to uracil whereas other modified cytosines (e.g., 5-methylcytosine, 5-hydroxylmethylcystosine) are not converted. Performing bisulfite conversion can facilitate identifying positions containing mC or hmC using the sequence reads. For an exemplary description of bisulfite conversion, see, e.g., Moss et al., Nat Commun. 2018; 9:5068.

Examples of such conversion techniques also include oxidative bisulfite (Ox-BS) conversion. Performing Ox-BS conversion can facilitate identifying positions containing mC using the sequence reads. For an exemplary description of oxidative bisulfite conversion, see, e.g., Booth et al., Science 2012; 336:934-937.

Examples of such conversion techniques also include Tet-assisted bisulfite (TAB) conversion. For example, as described in Yu et al., Cell 2012; 149:1368-80, ß-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected. Thus, when TAB conversion is used, the first nucleobase comprises one or more of unmodified cytosine, fC, caC, mC, or other cytosine forms affected by bisulfite, and the second nucleobase comprises hmC. Performing TAB conversion can facilitate identifying positions containing hmC using the sequence reads.

Examples of such conversion techniques also include Tet-assisted conversion with a substituted borane reducing agent, optionally wherein the substituted borane reducing agent is 2-picoline borane, borane pyridine, tert-butylamine borane, or ammonia borane. See, e.g., Liu et al., Nature Biotechnology 2019; 37:424-429 (e.g., at Supplementary FIG. 1 and Supplementary Note 7). Performing TAP conversion can facilitate identifying positions containing unmodified C using the sequence reads. This procedure encompasses Tet-assisted pyridine borane sequencing (TAPS), described in further detail in Liu et al. 2019, supra.

Alternatively, protection of hmC (e.g., using BGT) can be combined with Tet-assisted conversion with a substituted borane reducing agent. Performing such TAPSß conversion can facilitate distinguishing positions containing unmodified C or hmC on the one hand from positions containing mC using the sequence reads. For an exemplary description of this type of conversion, see, e.g., Liu et al., Nature Biotechnology 2019; 37:424-429.

Examples of such conversion techniques also include APOBEC-coupled epigenetic (ACE) conversion. Performing ACE conversion can facilitate distinguishing positions containing hmC from positions containing mC or unmodified C using the sequence reads. For an exemplary description of ACE conversion, see, e.g., Schutsky et al., Nature Biotechnology 2018; 36:1083-1090.

Examples of such conversion techniques also include enzymatic conversion of a nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1.

In some embodiments, methylation is detected using a methylation-sensitive restriction enzyme (MSRE). For example, a portion of a sample can be subjected to digestion with one or more MSREs to cleave unmethylated sequences. Exemplary MSREs include AatII, AccII, Acil, Aor13HI, Aor15HI, BspT104I, BssHII, BstUI, Cfr10I, Clal, Cpol, Eco52I, HaeII, HapII, HhaI, Hin6I, HpaII, HpyCH4IV, MluI, MspI, Nael, NotI, Nrul, NsbI, PmaCI, Psp1406I, Pvul, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, Hpall and Hin6I. In some embodiments, the portion of the sample that is contacted with one or more MSREs comprises hypermethylated DNA, or is or comprises a hypermethylated DNA partition, which may be obtained as described elsewhere herein.

In some embodiments, DNA fragmentation is detected by determining the endpoints and/or midpoints of sequenced fragments of DNA (e.g., cfDNA). For example, differences in fragmentation patterns may occur depending on whether the fragments originated from a tumor or from healthy cells.

### E. Adapter Ligation or Addition; Tagging

In some embodiments, the disclosed methods further comprise analyzing DNA in a sample (which may be a separate sample from the same subject or the same sample). In such methods, adapters may be added to the DNA. This may be done concurrently with an amplification procedure, e.g., by providing the adapters in a 5′ portion of a primer (where PCR is used, this can be referred to as library prep-PCR or LP-PCR). In some embodiments, adapters are added by other approaches, such as ligation. In some such methods, prior to partitioning or prior to capturing, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA is partitioned, comprising contacting the DNA with an agent that preferentially binds to nucleic acids bearing an epigenetic modification. The nucleic acids are partitioned into at least two subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

Tagging DNA molecules is a procedure in which a tag is attached to or associated with the DNA molecules. Such tags can be molecules, such as nucleic acids, containing information that indicates a feature of the molecule with which the tag is associated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. Tags can be used to label the individual polynucleotide population partitions so as to correlate the tag (or tags) with a specific partition. Alternatively, tags can be used in embodiments that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Molecular tagging refers to a tagging practice that allows one to differentiate among DNA molecules from which sequence reads originated. Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by U.S. patent applications 20010053519, 20030152490, 20110160078, and U.S. Pat. Nos. 6,582,908 and 7,537,898 and 9,598,731. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

### F. Enriching/Capturing Step; Amplification

Methods disclosed herein can comprise enriching, capturing, or isolating complexes, such as complexes comprising cell debris and target molecules, and/or enriching, capturing, or isolating DNA, such as cfDNA target regions. In some embodiments, the capturing comprises contacting the complexes or target molecules with binding molecules specific for a cell debris marker and/or an exosome marker, or a target molecule and/or contacting the DNA with probes specific for target regions. Enrichment or capture may be performed on any sample or subsample described herein using any suitable approach known in the art.

In some embodiments, the binding molecules specific for markers or target molecules or the probes specific for DNA target regions comprise a capture moiety that facilitates the enrichment or capture of target molecules or the DNA hybridized to the probes, respectively. In some embodiments, the capture moiety is biotin. In some such embodiments, streptavidin attached to a solid support, such as magnetic beads, is used to bind to the biotin. In some embodiments, nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, the methods herein comprise enriching for or capturing DNA comprising epigenetic and/or sequence-variable target regions. Such regions may be captured from an aliquot of a sample (e.g., a sample that has undergone attachment of adapters and amplification), while the step of partitioning the DNA with an agent that recognizes methyl cytosine is performed on a separate aliquot of the sample. Enriching for or capturing DNA comprising epigenetic and/or sequence-variable target regions may comprise contacting the DNA with a first or second set of target-specific probes. Such target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample or the second subsample, e.g., the first subsample and the second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject. The target regions may comprise differences depending on whether they originated from a tumor or from healthy cells or from a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, the DNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured DNA, e.g., to different degrees of sequencing depth for the epigenetic and sequence-variable target region sets, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

### G. Captured Set; Target Regions

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell types but not to the extent observed in the one cell type. In some embodiments, the hypermethylation variable target regions show lower methylation in healthy cfDNA than in at least one other tissue type.

In some embodiments, a captured epigenetic target region set captured from a sample or second subsample comprises hypomethylation variable target regions. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell types but not to the extent observed in the one cell type. In some embodiments, the hypomethylation variable target regions show higher methylation in healthy cfDNA than in at least one other tissue type.

Without wishing to be bound by any particular theory, in an individual with cancer, proliferating or activated immune cells and/or cancer cells may shed more DNA into the bloodstream than immune cells in a healthy individual and/or healthy cells of the same tissue type, respectively. As such, the distribution of cell type and/or tissue of origin of cfDNA may change upon carcinogenesis. Thus, variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole gnome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set. The sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNEI, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKTI, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1.

In some embodiments, the sequence-variable target region set comprises target regions from at least 10, 20, 30, or 35 cancer-related genes, such as the cancer-related genes listed above and in Tables 3 and 4 of WO 2020/160414.

### H. Sequencing

In general, sample proteins and/or nucleic acids, including nucleic acids flanked by adapters, with or without prior amplification can be subject to sequencing. Sequencing methods include, for example, Edman degradation based protein sequencing, mass spectrometry based protein sequencing, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms.

In some embodiments, sequencing comprises detecting and/or distinguishing unmodified and modified nucleobases. For example, single-molecule real-time (SMRT) sequencing facilitates direct detection of, e.g., 5-methylcytosine and 5-hydroxymethylcytosine as well as unmodified cytosine. See, e.g., Schatz., Nature Methods. 14 (4): 347-348 (2017); and U.S. Pat. No. 9,150,918. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can performed on at least 5, 10, 20, 70, 100, 200 or 500 different genes, or at most 5000, 2500, 1000, 500 or 100 different genes.

Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

### III. Additional Features of Certain Disclosed Methods

### A. Samples

A sample can be any biological sample isolated from a subject. A sample can be a bodily sample. Samples can include body tissues or fluids, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, gingival crevicular fluid, bone marrow, pleural effusions, pleura fluid, cerebrospinal fluid, saliva, mucous, sputum, semen, sweat, and urine. Samples are preferably body fluids, particularly blood and fractions thereof, cerebrospinal fluid, pleura fluid, saliva, sputum, or urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

### B. Capture Moieties

As discussed above, molecules, such as proteins and/or nucleic acids in a sample can be subject to a capture step, in which target molecules or molecules having target regions are captured and analyzed. Target capture can involve use of oligonucleotides labeled with a capture moiety, such as biotin, and a second moiety or binding partner that binds to the capture moiety, such as streptavidin. In some embodiments, a capture moiety and binding partner can have higher and lower capture yields for different sets of target regions, such as those of the sequence-variable target region set and the epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### C. Computer Systems

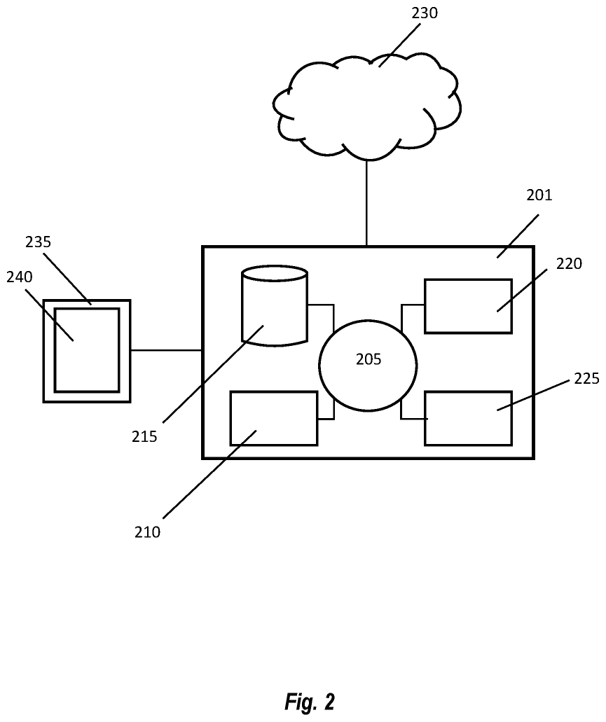

Methods of the present disclosure can be implemented using, or with the aid of, computer systems. FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including (where applicable) nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method described herein. For example, the method may comprise: collecting a sample from a subject and, optionally, fractionating the sample; contacting the sample or subsample thereof with at least one cell debris marker binding molecule; capturing complexes comprising the cell debris marker binding molecule or directly detecting target molecules associated with the complexes; detecting and identifying the levels of target molecules the likelihood that the subject has cancer or another disease and/or an appropriate treatment for the cancer of other disease.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5^{th} Ed. (2011), Kurose, Computer Networking: A Top-Down Approach, Pearson, 7^{th}) Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### D. Applications

### 1. Cancer and Other Diseases

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of target molecules, copy number variation, or rare mutations detected in subject's blood if the treatment is successful as more cancers may die and shed cell debris. In other examples, this may not occur. In another example, certain treatment options may be correlated with profiles (e.g., of cell-debris associated proteins and/or genetic profiles) of cancers over time. This correlation may be useful in selecting a therapy.

In some embodiments, the present methods are used for screening for a cancer, or in a method for screening cancer. For example, the sample can be from a subject who has not been previously diagnosed with cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at www.dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the presence of one or more proteins of interest and/or classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Target molecule and genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular molecules and polynucleotides derived from the subject, wherein the genetic profile comprises a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate a profile, fingerprint or set of data that is a summation of target molecule and genetic information derived from different cells in a heterogeneous disease. This set of data may comprise copy number variation, epigenetic variation, and mutation analyses alone or in combination.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of a target molecule associated with cell debris originating or derived from a tumor cell at a preselected timepoint following a previous cancer treatment of a subject previously diagnosed with cancer. DNA originating or derived from the tumor cell may also be detected. The method may further comprise determining a cancer recurrence score that is indicative of the presence or absence of the target molecule and, where applicable, DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Subject and/or Classifying a Subject as being a Candidate for a Subsequent Cancer Treatment

In some embodiments, a method provided herein is a method of determining a risk of cancer recurrence in a subject. In some embodiments, a method provided herein is a method of classifying a subject as being a candidate for a subsequent cancer treatment.

Any of such methods may comprise collecting a sample from the subject diagnosed with the cancer at one or more preselected timepoints following one or more previous cancer treatments to the subject. The subject may be any of the subjects described herein. The sample may comprise cell debris. The sample may comprise DNA, e.g., cfDNA. The DNA may be obtained from a tissue sample.

Any of such methods may comprise contacting the sample or a subsample thereof with at least one binding molecule and detecting the presence or level of at least one target molecule according to any of the embodiments as described herein. The methods may further comprise capturing a plurality of sets of target regions from DNA from the subject, wherein the plurality of target region sets comprise a sequence-variable target region set, and/or an epigenetic target region set, whereby a captured set of DNA molecules is produced. The capturing step may be performed according to any of the embodiments described elsewhere herein. Any of such methods may comprise sequencing the captured DNA molecules, whereby a set of sequence information is produced. The captured DNA molecules of a sequence-variable target region set may be sequenced to a greater depth of sequencing than the captured DNA molecules of the epigenetic target region set. Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

In any of such methods, the previous cancer treatment may comprise surgery, administration of a therapeutic composition, and/or chemotherapy.

Methods of determining a risk of cancer recurrence in a subject may comprise determining a cancer recurrence score that is indicative of the presence or absence, or amount, of at least one target molecule and/or the DNA originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises sequence-variable target region sequences and determining the cancer recurrence score may comprise determining at least a first subscore indicative of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Therapies and Related Administration

In certain embodiments, the methods disclosed herein relate to identifying and administering customized therapies to patients. In some embodiments, determination of the levels of particular target molecules or cell debris facilitates selection of appropriate treatment. In some embodiments, the patient or subject has a given disease, disorder or condition. Essentially any cancer therapy (e.g., surgical therapy, radiation therapy, chemotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

In some embodiments, e.g., where genetic variants are detected, therapy is customized based on the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### IV. Kits

Also provided are kits comprising the compositions as described herein. The kits can be for use in performing the methods as described herein. In some embodiments, a kit comprises one or more cell debris marker binding molecules. In some embodiments, the kit comprises an exosome marker binding molecule, e.g., in addition to the one or more cell debris marker binding molecules. In some embodiments, the marker binding molecule comprises a label or capture moiety. In some embodiments, the kit comprises a solid support linked to a binding partner of the capture moiety. In some embodiments, the kit comprises one or more target molecule binding molecules. In some embodiments, the kit comprises reagents for detecting the presence or levels of target molecules.

In some embodiments, a kit further comprises an agent that recognizes methyl cytosine in DNA. In some such embodiments, the agent is an antibody or a methyl binding protein or methyl binding domain. In some embodiments, the kit comprises target-specific probes that specifically bind to epigenetic and/or sequence-variable target region sets. In some such embodiments, the target-specific probes comprise a capture moiety. In some embodiments, the kit comprises a solid support linked to a binding partner of the capture moiety. In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNFIA, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNEI, ESR1, R1T1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the library adapters can be found throughout and are applicable to the kit.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Analysis of Circulating Proteins Using Simultaneous Detection of a Target Protein and of an Exosome Marker

A set of patient samples are analyzed by a blood-based assay to detect the presence/absence of cancer. First portions of whole blood samples of these patients are fractionated via centrifugation at 1,600 g for 10 minutes at 10° C. into plasma, buffy coat, and red blood cell fractions. The plasma fractions are further fractionated via centrifugation at 3,220 g for 10 minutes at 10° C. to produce plasma pellets and plasma supernatants. The buffy coat fractions, plasma pellets, and second portions of the whole blood samples are contacted with a first antibody that specifically binds phosphatidylserine conjugated to a first oligonucleotide and with a second antibody that specifically binds a target molecule, such as CTLA4 or PDL1, conjugated to a second oligonucleotide. Optionally, the plasma supernatants are contacted with a first antibody that specifically binds an exosome marker conjugated to a first oligonucleotide and with a second antibody that specifically binds a target molecule, such as CTLA4 or PDL1, conjugated to a second oligonucleotide. Each of the first and second oligonucleotides comprises two portions: 1) a first portion having a sequence that is unique to the antibody to which it is conjugated (molecular barcode) and 2) a second portion, 3′ relative to the first portion, having a hybridization sequence. The hybridization sequences of the first oligonucleotides, which are the same as each other, are complementary to the hybridization sequence of each second oligonucleotide. If the first and second oligonucleotides are within close enough proximity to each other, they will hybridize, and hybridized (double-stranded) oligonucleotide sequences are extended from the 3′ ends of the hybridization sequences using a DNA polymerase. The extended oligonucleotides are pooled, amplified, and sequenced using an Illumina sequencer or quantified by a suitable procedure, such as qPCR. For an exemplary hybridization, extension, and sequencing-based detection procedure, see, e.g., Gong et al., Bioconjugate Chem. 2016, 27, 1, 217-225.

The sequence reads generated by the sequencer are then analyzed using bioinformatic tools/algorithms. The molecular barcodes present in the sequenced molecules are used to identify antibodies and their antigen molecules while also deconvoluting the cell debris or, if applicable, exosome marker to which they were in proximity. Quantification of the sequence reads that correspond to proteins, such as proteins upregulated in tumor cells, in a sample from a patient relative to a sample from a healthy subject facilitates a determination of the likelihood that the patient has cancer.

### Example 2: Analysis of Circulating Proteins Using Sequential Detection of a Target Protein and of an Exosome Marker

A set of patient whole blood samples are fractionated as described in Example 1. The buffy coat fractions, plasma pellets, and second portions of the whole blood samples are contacted with Annexin V conjugated to biotin, then contacted with magnetic beads conjugated to streptavidin to isolate cell debris. Optionally, the plasma supernatants are contacted with an exosome marker binding molecule conjugated to biotin, then contacted with magnetic beads conjugated to streptavidin to isolate exosomes. The beads and molecules bound thereto are precipitated, and any unbound sample components are washed away from the beads with buffers containing increasing concentrations of salt. A high salt buffer is used to wash the cell debris away from the Annexin V and, if applicable, the exosomes away from the exosome marker binding molecule. The precipitated, enriched molecules are cleaned, to remove salt, and concentrated in preparation for target molecule detection steps.

If the identities of the target molecules are known, the isolated cell debris and exosomes, if applicable, are contacted with antibodies for the target molecules and detected using an immunoassay or flow cytometry. If the identities of the target molecules are known or unknown, the proteins are purified from the membrane and other material of the cell debris and exosomes, if applicable, and the proteins are analyzed by mass spectrometry to identify and/or quantify them.

### Example 3: Combination Analysis of Circulating Proteins and cfDNA Using Sequential Detection of a Target Protein and of an Exosome Marker

A set of patient whole blood samples are fractionated as described in Example 1. The buffy coat fractions, plasma pellets, and second portions of the whole blood samples are treated and analyzed as described in Example 2. The plasma supernatants are divided into multiple aliquots. First aliquots are treated and analyzed as described in Example 2. cfDNA is extracted from second aliquots.

cfDNA of the subject samples is then partitioned based on cytosine methylation levels. The cfDNA is contacted with an antibody that recognizes methyl cytosine, then immunoprecipitated using magnetic beads conjugated to protein G, thus partitioning hypermethylated DNA from hypomethylated DNA. Any non-methylated or less methylated DNA is washed away from the beads with buffers containing increasing concentrations of salt. Finally, a high salt buffer is used to wash the heavily methylated DNA away from the antibody to provide a hypermethylated partition, an intermediate partition, and a hypomethylated partition.

After concentrating the cfDNA in the partitions, first adapters are added to the cfDNA by ligation to the 3′ ends thereof. The adapter is used as a priming site for second-strand synthesis using a universal primer and a DNA polymerase. The first adapter comprises a biotin, and nucleic acid ligated to the first adapter is bound to beads comprising streptavidin. A second adapter is then be ligated to the 3′ end of the second strand of the now double-stranded molecules. These adapters contain non-unique molecular barcodes, and each partition is ligated with adapters having non-unique molecular barcodes that is distinguishable from the barcodes in the adapters used in the other partitions. After ligation, the partitions are pooled together and are amplified by PCR.

Following PCR, amplified DNA is washed and concentrated prior to enrichment. Once concentrated, the amplified DNA is combined with a salt buffer and biotinylated RNA probes that comprise probes for a sequence-variable target region set and probes for an epigenetic target region set and this mixture is incubated overnight. The probes for the sequence-variable region set have a footprint of about 50 kb and the probes for the epigenetic target region set has a footprint of about 500 kb. The probes for the sequence-variable target region set comprise oligonucleotides targeting at least a subset of genes described herein and the probes for the epigenetic target region set comprises oligonucleotides targeting a selection of hypermethylation variable target regions, hypomethylation variable target regions, and optionally one or more of CTCF binding target regions, transcription start site target regions, focal amplification target regions and methylation control regions.

The biotinylated RNA probes (hybridized to DNA) are captured by streptavidin magnetic beads and separated from the amplified DNA that are not captured by a series of salt-based washes, thereby enriching the sample. After enrichment, an aliquot of the enriched sample is sequenced using Illumina NovaSeq sequencer. The sequence reads generated by the sequencer are then analyzed using bioinformatic tools/algorithms. The molecular barcodes are used to identify unique molecules as well as for deconvolution of the sample into molecules that were differentially partitioned. The method described in this example, apart from providing information on the overall level of methylation (i.e., methylated cytosine residues) of a molecule based on its partition, can also provide a higher resolution information about the identity and/or location of the type of methylated cytosine. The sequence-variable target region sequences are analyzed by detecting genomic alterations such as SNVs, insertions, deletions and fusions that can be called with enough support that differentiates real tumor variants from technical errors (for e.g., PCR errors, sequencing errors). The epigenetic target region sequences are analyzed independently to detect methylated cfDNA molecules in regions that have been shown to be differentially methylated in cancer compared to normal cells. Finally, the results of the analyses are combined to produce a final tumor present/absent call.

1. A method of detecting a cell debris-associated target molecule in a sample, the method comprising:
a) contacting the sample or a subsample thereof with at least one binding molecule, wherein the at least one binding molecule binds a cell debris marker, thereby producing complexes comprising the at least one binding molecule and cell debris, the cell debris comprising a membrane fragment; and
b) detecting the presence or level of at least one target molecule associated with the complexes.

2. The method of claim 1, wherein the sample is obtained from a subject and/or wherein the sample is a blood sample.

3. (canceled)

4. The method of claim 2, wherein the blood sample is
(i) a whole blood sample;
(ii) a plasma sample; or
(iii) a plasma pellet sample or a buffy coat sample.

5. (canceled)

6. (canceled)

7. The method of claim 1, wherein
(i) the at least one binding molecule is a protein, wherein the protein is optionally an antibody; and/or
(ii) the cell debris marker is an inner membrane marker, and/or the cell debris marker is phasphatidylserine or phosphatidylethanolamine.

8. (canceled)

9. (canceled)

10. (canceled)

11. The method of claim 1, wherein:
(i) the cell debris marker is phosphatidylserine and the at least one binding molecule is Annexin V or an antibody specific for phosphatidylserine; or
(ii) the cell debris marker is phosphatidylethanolamine, and the at least one binding molecule is an antibody specific for phosphatidylethanolamine.

12. (canceled)

13. (canceled)

14. (canceled)

15. The method of claim 1, wherein the at least one binding molecule comprises a label or is conjugated to a solid support.

16. The method of claim 15, wherein
(i) the at least one binding molecule comprises a label and the method further comprises capturing the at least one binding molecule by binding the label to a solid support;
(ii) the at least one binding molecule is conjugated to a label, wherein the label comprises a fluorophore, biotin, a peptide, or an oligonucleotide; and/or
(iii) the solid support comprises a bead, optionally wherein the at least one binding molecule is conjugated to a magnetic bead.

17. (canceled)

18. (canceled)

19. (canceled)

20. The method of claim 1, wherein the method comprises capturing the complexes from the sample or subsample thereof prior to the detecting, optionally wherein the capturing comprises separating components of the sample or subsample thereof that are not bound to the at least one binding molecule from the complexes to which the at least one binding molecule is bound.

21. (canceled)

22. The method of claim 20, wherein the detecting comprises mass spectrometric analysis of target molecules associated with the complexes.

23. The method of claim 20, wherein the detecting comprises contacting the complexes with at least one binding molecule that binds a target molecule potentially associated with the complexes, optionally wherein at least one binding molecule that binds a target molecule associated with the complexes is an antibody specific for a target molecule.

24. (canceled)

25. The method of claim 23, wherein at least one binding molecule that binds a target molecule comprises a label, optionally wherein the label is a fluorophore or an oligonucleotide.

26. (canceled)

27. The method of claim 25,
wherein the label is an oligonucleotide and the binding molecule that binds a cell debris marker comprises an oligonucleotide, and/or
wherein the detecting comprises a proximity ligation assay or a proximity extension assay.

28. (canceled)

29. (canceled)

30. The method of claim 20, wherein the detecting comprises:
(i) an immunoassay, optionally wherein the immunoassay is an enzyme-linked immunosorbent assay, a sandwich assay, an electrochemiluminescent assay, or a multiplex immunoassay; and/or
(ii) flow cytometric analysis of the complexes.

31. (canceled)

32. (canceled)

33. The method of claim 1, wherein a plurality of target molecules associated with the complexes are detected, optionally wherein the plurality of target molecules is 2 to 10,000, 2 to 5,000, 2 to 1,000, or 2 to 100 target molecules.

34. (canceled)

35. The method of claim 1, wherein the method comprises capturing the complexes after contacting the complexes with at least one binding molecule that binds a target molecule potentially associated with the complexes.

36. The method of claim 33, wherein;
(i) at least one target molecule, two or more of the plurality of target molecules, or each of the plurality of target molecules is a protein;
(ii) at least one target molecule, two or more of the plurality of target molecules, or each of the plurality of target molecules is a carbohydrate, optionally a glycoprotein carbohydrate;
(iii) at least one target molecule is a molecule associated with a disease, two or more of the plurality of target molecules is a molecule associated with a disease, or each of the plurality of target molecules is a molecule associated with a disease; and/or
(iv) at least one target molecule, two or more target molecules, or each of the plurality of target molecules is a cell type marker.

37. (canceled)

38. (canceled)

39. The method of claim 36,
wherein the disease is cancer and wherein the at least one target molecule is upregulated in tumor cells relative to healthy cells of the same tissue type; and/or
wherein at least one, two or more, or each of the target molecules is selected from PD-L1, CTLA4, NYESO1, mesothelin, CA15-3, CA19-9, CA-125, and CA-172-4.

40. (canceled)

41. (canceled)

42. The method of claim 36, wherein at least one target molecule, two or more target molecules, or each of the plurality of target molecules is a cell type marker, optionally wherein the cell type markers are selected from:
(i) markers for immune cells and solid tissue cells; and/or
(ii) markers for colon, lung, breast, skin, prostate, stomach, pancreas, and liver cell type markers.

43. (canceled)

44. (canceled)

45. The method of claim 1, wherein the sample is obtained from a subject having a disease, and the detecting comprises identifying a plurality of target molecules, wherein the identifying comprises mass spectrometric analysis of target proteins.

46. The method of claim 1, wherein the method comprises:
(i) measuring total cell debris levels in the sample or subsample thereof, optionally wherein the sample is obtained from a subject having a disease, and wherein the total cell debris levels is measured relative to total cell debris levels in a sample or subsample thereof obtained from a healthy individual; and/or
(ii) analyzing DNA in a subsample of the sample or in a second sample obtained from the same subject from which the first sample is obtained, optionally wherein the subsample or second sample is a plasma or serum sample and/or wherein the DNA is cfDNA.

47. (canceled)

48. (canceled)

49. (canceled)

50. (canceled)
